(19) United States
(12) Patent Application Publication
(10) Pub. No.: US 2024/0378765 A1
(43) Pub. Date: Nov. 14, 2024

(54) SYSTEMS AND METHODS FOR CONVOLUTIONAL NEURAL NETWORK-BASED IMAGE SYNTHESIS

(71) Applicant: Idealab Studio, LLC, Pasadena, CA (US)

(72) Inventors: William Tod Gross, Pasadena, CA (US); Andrea Pedretti, Thousand Oaks, CA (US)

(21) Appl. No.: 18/659,295

(22) Filed: May 9, 2024

Related U.S. Application Data
(60) Provisional application No. 63/501,342, filed on May 10, 2023.

(51) Int. Cl. G06T 11/00; A61B 8/00; A61B 8/08; B25J 19/02; G01S 7/41; G01S 13/86; G06T 7/00; G16H 30/40

(52) U.S. Cl. CPC G06T 11/00; A61B 8/4218; A61B 8/5207; A61B 8/5261; B25J 19/021; B25J 19/026; G01S 7/417; G01S 13/862; G01S 13/865; G06T 7/97; G16H 30/40; G06T 2207/20081; G06T 2207/20084; G06T 2207/30004

(57) ABSTRACT

Methods and systems are disclosed configured to access a learning engine trained to generate an inferred image from a combination of reflections of different signal types, the different signal types comprising WiFi, light, and/or sound signals. WiFi, light, and/or sound signals are caused to be directed at a subject from a WiFi transmitter; a light emitter, and a sound generator, respectively. Respective receivers are used to receive reflections of the WiFi, light, and/or sound signals from an object. The trained learning engine may be utilized to generate an inferred image of an internal structure of the object from the received reflections of the WiFi, light, and/or sound signals.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### COPYRIGHT NOTICE

A portion of the disclosure of this patent document contains material which is subject to copyright protection. The copyright owner has no objection to the facsimile reproduction by anyone of the patent document and/or the patent disclosure as it appears in the United States Patent and Trademark Office patent file and/or records, but otherwise reserves all copyrights whatsoever.

### BACKGROUND OF THE INVENTION

### Field of the Invention

The present disclosure generally relates to image synthesis using a learning engine.

### Description of the Related Art

Medical examinations often utilize invasive imaging techniques such as x-rays and ultrasound to help diagnose and monitor various health conditions.

X-ray imaging use electromagnetic radiation to create images of the inside of the body. They are commonly used to examine bones and teeth, as they can easily penetrate these hard tissues. X-rays can also be used to look for abnormalities in soft tissues, such as the lungs, breasts, and digestive system. Disadvantageously, repeated exposure to x-rays can increase the risk of cancer, and so repeated use may result in serious medical issues.

Ultrasound imaging uses high-frequency sound waves to create images of the inside of the body. Ultrasound is commonly used to examine the uterus and ovaries during pregnancy, as well as to evaluate the heart, liver, and other organs. Disadvantageously, there may be discomfort or pain associated with the procedure, particularly if an ultrasound probe is inserted into a body cavity.

Disadvantageously, both X-rays and ultrasound imaging involve expensive equipment that, generally only available at medical facilities.

### BRIEF DESCRIPTION OF THE DRAWINGS

Embodiments will now be described with reference to the drawings summarized below. These drawings and the associated description are provided to illustrate example aspects of the disclosure, and not to limit the scope of the invention.

FIG. 1 illustrates an example architecture.

FIG. 2A illustrates an example process.

FIG. 2B illustrates an example convolutional neural network.

FIG. 3 illustrates an example inspection enclosure.

FIGS. 4 and 5 illustrate example processes.

FIG. 6 illustrates an example environment for generating an inferred image.

FIGS. 7A-7D illustrate example implementations.

### DETAILED DESCRIPTION

An aspect of the present disclosure relates to systems and methods for utilizing artificial intelligence to generate artificial images from non-invasive, low risk wavelengths of radio signals, sound signals, and/or optical signals. Such artificial images may be utilized to determine a change in state and/or may be used to identify a medical event. In addition, the disclosed systems and methods may be utilized for non-medical applications, such as to detect the presence of objects behind a wall or that are otherwise not optically visible.

An artificial intelligence learning engine may be trained to convert one or more signal reflections from an object (such as a limb or other animate or inanimate object) into an inferred (sometimes referred to as a predicted or artificial) image. Optionally, different learning engines may be trained for different object-types (e.g., live objects, inanimate objects, different limbs, different age groups, etc.). Optionally, a learning engine may be trained to be usable for several or many object types.

For example, the learning engine may receive reflected RF (e.g., WiFi), sound, and/or light signals and generate an artificial image from such signals. The artificial image may optionally be of relatively low resolution and/or may not be precise or a fully accurate representation of the object. For example, an artificial image of an interior structure of a limb (e.g., muscles, tissue, bones, etc.) may be sufficient to determine that some type of medical issue exists, even if the image is not sufficient to determine the precise nature of the issue or to perform a diagnosis. Optionally, if it is determined that some type of medical issue exists, the person can then go to a medical facility to have an actual image captured (e.g., via an x-ray, MRI, etc.) and analyzed (e.g., by a radiologist), or to be otherwise examined.

For example, the learning engine may comprise one or more neural networks trained to synthesize/predict an image from some combination of RF, acoustic signals, and/or light signals (which may be referred to multi-signal type signals) via signal-to-image synthesis. The artificial intelligence learning engine may be trained using supervised or unsupervised training.

For example, the learning engine may be trained to learn the relationship between multi-signal type signals and corresponding images using training data sets. By way of illustration, the learning engine may receive multi-signal type signals corresponding to reflected signals from an object, attempt to generate an inferred image of the object, and then may be provided with an actual image of the object. The learning engine may generate an error value based on the comparison of the actual image with the inferred image, and may then use the error value to adjust certain learning image parameters to better create a matching image. For example if the learning engine comprises a neural network, the neural network layer node weights may be accordingly adjusted using backpropagation based on an error function output with respect to the comparison of the actual object image and the inferred object image generated by the neural network to thereby lower the error.

Optionally, the neural network may be composed of a multi-signal type signal encoder and an image decoder. The multi-signal type signal encoder may receive the input multi-signal type signals, such as signal reflections from an object (e.g., from the internal structure of a human or animal limb, or from an inanimate object), and convert signals into a numerical representation. The image decoder may input this numerical representation and generate an image that corresponds to the multi-signal type signals.

Optionally, the multi-signal type signals may undergo preprocessing, then image synthesis may be performed to extract meaningful features that can be used to generate an image. For example, with respect to acoustic/sound signals, this may involve techniques such as Fourier analysis and/or Mel-frequency cepstral coefficients (MFCCs) to transform acoustic into a format that can be used by the machine learning algorithm. By way of further example, with respect to RF signals, the reflected RF signals may optionally be pre-processed to extract relevant features, such as the amplitude, frequency, and phase of the signal.

A machine learning algorithm, such as a convolutional neural network (CNN), receives the preprocessed multi-signal type signals as input and generates an image as output. The generated, inferred image may be a direct representation of the multi-signal type signal waveform, or it may be a more abstract representation that captures the essence of the multi-signal type signal.

Additional, optional postprocessing may be performed on the inferred image to enhance its visual quality or to better match the input multi-signal type signal. This may involve techniques such as image filtering or blending to create a more visually appealing result. Other optional example postprocessing steps are described elsewhere herein.

By way of example the convolutional deep neural network may be configured with a shared-weights architecture and with translation invariance characteristics. The neural network may include an input layer, an output layer, and one or more hidden layers. The hidden layers may be configured as convolutional layers, pooling layers, fully connected layers and/or normalization layers. For example, the convolutional deep neural network may be configured with pooling layers that combine outputs of neuron clusters at one layer into a single neuron in the next layer. Max pooling and/or average pooling may be utilized. Max pooling may utilize the maximum value from each of a cluster of neurons at the prior layer. Average pooling may utilize the average value from each of a cluster of neurons at the prior layer.

Certain aspects will now be described with reference to the figures.

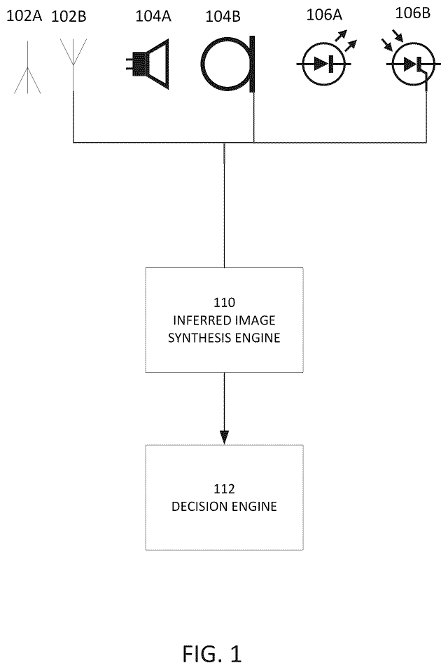

FIG. 1 illustrates an example architecture including one or more sensor sets. A sensor set may include multiple types of sensors. The sensor set may optionally be configured with emitters that emit signals that cause little or no risk to a subject that is the target of the signals. Hence, advantageously, there may be no need to track the amount or number of exposures of a subject to the signals (although such tracking may still be optionally performed). In this example, the sensor set includes signal emitters and signal reflection receivers. The example illustrated sensor set includes an RF emitter 102A (e.g., a WiFi transmitter), an RF receiver 102B (e.g., a WiFi receiver), an acoustic emitter 104A (e.g., an array of piezoelectric crystals, a cone speaker, a ribbon speakers, or other transducer), an acoustic receiver 104B (e.g., a microphone configured to receive reflections of sound waves from the acoustic emitter 104A), a light emitter 106A (e.g., light emitting diodes, incandescent bulbs, etc.), and a light sensor 106B (e.g., a photocell).

There may be more than one emitter and/or one receiver of each sensor type. Optionally, the number of signal emitters may be different than the number of signal receivers. For example, there may be two or three receivers for a corresponding emitter. The receivers and/or emitters of a given type may be linearly and/or radially distributed along or about a given subject. Optionally, a given emitter and/or receiver may be mounted on a transport mechanism (e.g., a motorized linear or spiral actuator) so that they may be moved across or around a subject, taking perioding snapshots of signal reflections from the subject at different points of the subject. Optionally, one or more sensors (e.g., ultrasound emitters and/or receivers) may be mounted on one or more robotics arms (e.g., removably mounted at a distal end of a robotic arm). The robotic arms may be configured to identify and/or track a subject. The positioning and pose of the robotic arms may be controlled by a control system and/or by a human.

For example, a first robotic arm with an emitter may be positioned on one side of a subject, and a second robotic arm with a receiver may be positioned on a second side of a subject. The two arms may be moved in unison across the subject so as to capture signals passing through the subject. The pose of the emitter and/or receiver may be adjusted by the control system to ensure that the emitted signals may be received by the receiver. The received signals may then be used to generate an image. Thus, for example, the disclosed system may be configured to perform ultrasound tomography.

Optionally, one or more large emitters may be positioned on a robot arm that scans the entire subject by emitting signals across the subject, and fixed receivers (e.g., positioned in a bathtub or other liquid container) positioned on the other side of the subject may be utilized to receive the emitted signals passing through the subject. Optionally, one or more fixed emitters may be positioned on one side of the subject and one or more receivers may be positioned on a robot arm that scans the entire subject to receive the emitted signals passing through the subject.

Each signal snapshot may optionally be timestamped and stored in association with position information (e.g., the distance from an initial point of travel). Optionally, a marker may be placed at one or more points on the subject that provides a unique or distinct reflection so as to be able to determine which area of the subject a given snapshot corresponds to. For example, the marker may be reflective ink drawn on the subject or a reflective sticker with adhesive on one side so as to adhere to the subject. The term “reflective” may comprise reflective with respect to RF, sound, and/or light signals.

The signal reflections received from the signal receivers may be provided to an inferred image synthesis engine 110. The inferred image synthesis engine may be configured to generate/predict an inferred image from the received reflected signals. The inferred image is not an optical image or intended to be a fully accurate image. Rather, it may be used to generally identify that an issue exists (e.g., a medical issue) and/or identify a subject-type (e.g., an automobile, a human arm, a couch, a suitcase, etc.). The inferred image synthesis engine 110 may comprise a preprocessor, an artificial intelligence learning engine (e.g., one or more neural networks), and a postprocessor. Optionally, the inferred image synthesis engine 110 may assign a category to the imaged subject (e.g., identifying the subject-type), which may be stored in association with the inferred image.

The inferred image output by the inferred image synthesis engine 110 (optionally in association with the identified subject-type) may be analyzed by a decision engine 112. The decision engine 112 may be utilized to determine if an action should be taken based at least in part on the inferred image. For example, if the decision engine 112 identifies some type of mass in a body part that is typically not found in that body part-type, the decision engine 112 may generate a notification comprising its finding and a link to, or the actual inferred image. The communication may be provided to medical personnel who may then schedule the subject for further examination and much better imaging.

Advantageously, the sensor components may optionally be very low cost (as compared to sensor components used in imaging equipment at medical facilities), and the inferred image synthesis engine 110 and/or the decision engine may be comprised of software (e.g., a mobile device application) that can execute on a user's mobile device. Hence, any ordinary user may be able to have the illustrated system in their home and may use the system as often as recommended or desired, without incurring medical costs. Further, a building, electrical, or plumbing contractor may utilize the illustrated system to determine what and where objects (e.g., studs, wiring, plumbing, beams, etc.) are within walls, floors, and/or ceilings. Optionally, the software may run on high speed processing devices, such as graphics processor units (GPUs).

A given GPU may comprise hundreds or thousands of processing cores that operate in parallel to process data in a highly efficient manner. A given core may comprise multiple streaming multiprocessors (SMs), configured to execute multiple instructions simultaneously. A given GPU may optionally be configured to perform geometry processing, including transforming and manipulating 3D models and other geometric objects to prepare them for rendering. A given GPU may optionally be configured to perform rasterization to convert geometric primitives into pixels that can be displayed on a display or that can be printed. A given GPU may optionally be configured to perform texturing, applying textures to the surfaces of 3D models. A given GPU may optionally be configured to perform lighting functions to simulate the behavior of light in a scene to create realistic shadows, reflections, and other effects. A given GPU may optionally be configured to perform compositing, comprising combining multiple layers of images and other visual elements to create a final image. Thus, a given GPU may be optimized to provide high-speed, high-quality rendering of a subject.

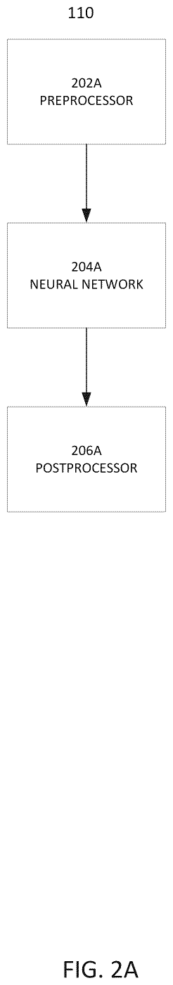

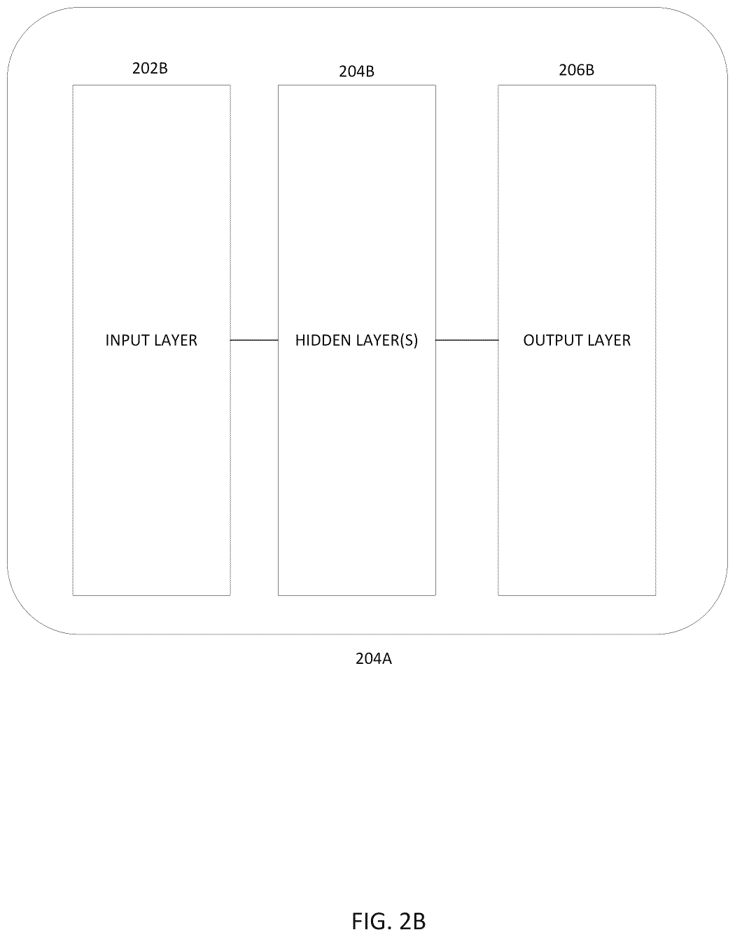

FIG. 2A illustrates the example image synthesis engine 110 in greater detail. The example image synthesis engine 110 comprises a preprocessor 202A, a neural network 204A, and a postprocessor 206A. For example, the preprocessor 202A may perform denoising, interpolation, registration, windowing, and/or normalization. In addition or instead, with respect to acoustic/sound signals, preprocessing may involve techniques such as Fourier analysis and/or Mel-frequency cepstral coefficients (MFCCs) to transform acoustic into a format that can be used by the neural network 204A. By way of further example, with respect to RF signals, the reflected RF signals may optionally be pre-processed by the preprocessor 202A to extract relevant features, such as the amplitude, frequency, and phase of the signal.

An example of the neural network 204A configured to generate inferred images from sensor inputs is illustrated in FIG. 2B. The neural network 204A may contain an input layer 202B, one or more hidden layers 204B, and an output layer 206B. The hidden layers 204B may be configured as convolutional layers, pooling layers, fully connected layers and/or normalization layers. For example, the neural network 204A may be configured with one or more pooling layers that combine outputs of neuron clusters at one layer into a single neuron in the next layer. Max pooling and/or average pooling may be utilized. Max pooling may utilize the maximum value from each of a cluster of neurons at the prior layer. Average pooling may utilize the average value from each of a cluster of neurons at the prior layer.

The neural network 204A may be trained based using a supervised or unsupervised process. For example, in supervised training, user feedback may be provided indicating how accurately the inferred image generated by the neural network 204A depicts the subject or internal structure thereof. The neural network layer node weights may be accordingly adjusted using backpropagation based on an error function output with respect to the accuracy of the inferred image generated by the neural network, to thereby lower the error.

Optionally, Generative Adversarial Networks (GANs) may be utilized to generate the inferred image. A GAN may comprise two neural networks: a generator and a discriminator. The generator takes random noise as input and produces an image that is intended to resemble the input image. The discriminator takes as input both the generated image and the original image (which may have been generated via more expensive, more accurate imaging systems, such as x-ray systems, MRI systems, and the like), and it attempts to distinguish between the two. The two networks are trained together in a process referred to as adversarial training.

During training, the generator tries to produce images that fool the discriminator into thinking they are real (rather than inferred images), while the discriminator tries to correctly distinguish between the generated images and the real images. As a result, the generator gradually learns to produce images that are increasingly similar to the original images while also satisfying the desired characteristics specified by the user.

Once the generator has been trained, it can be used to generate new images by providing it with random noise and the desired characteristics. The generator then produces an image that combines the characteristics with the random noise to produce a new, unique image.

The postprocessor 206A may perform contrast enhancement to improve the visibility of important features in the inferred image. For example, the brightness and contrast levels of the inferred image may be adjusted, and/or histogram equalization (where an image histogram provides a representation of the tonal distribution in the image) may be performed. In addition, optionally, the neural network 204A may generate a separate inferred image for each sensor type signal. Image fusion techniques may optionally be used to combine these different images into a single composite image, which can provide a more complete and accurate picture of the subject. In addition, in order to align images from different sensors or different time periods, image registration techniques may optionally be used. Such image registration techniques may comprise adjusting the position, rotation, and scaling of images to ensure that they line up correctly. Optionally, feature extraction techniques may be utilized to isolate certain features and enhance their visibility, making it easier to identify and analyze them.

A technical challenge in using the techniques disclosed herein with respect to generating artificial images (sometimes referred to herein as inferred images) by directing signals at a portion of human anatomy (e.g., an arm or a leg), is that signals of the desired frequency may bounce off the skin, and so will not penetrate to the internal structure. In order to overcome this technical challenge, an enclosure is described that may fit over a portion of the subject (e.g., a portion of a human anatomy), where the enclosure may be filled with a gel (aloe vera gel, which does not irritate the skin) and/or a liquid, such as, optionally, common, untreated, unfiltered tap water. The fluid or gel will provide a better interface between the signal emitters and the skin, enabling the signals to travel more easily through the skin and into the body. The signals may reflect off the body's internal structures, and the reflected signals may be received by a receiver which converts the received signals to a digital format suitable for an artificial intelligence component to generate inferred images based on the digitized signals.

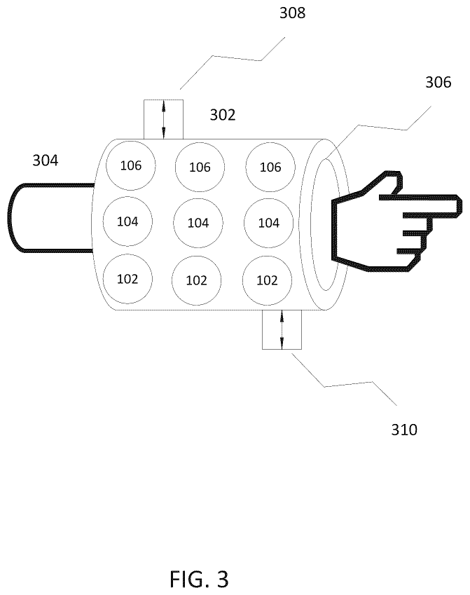

FIG. 3 illustrates an example enclosure 302, sized and configured to be utilized with respect to a human limb, such as an arm 304.

The enclosure 302 may be in the shape of a cylinder or cuboid and may be positioned on a human body portion in a vertical or horizontal orientation. The enclosure 302 may be in the form of a sleeve that can slide over a subject's arm 304 or leg. The sleeve may optionally be closed on one side and include a penetrable seal 306 on the other side comprising an entry opening (e.g., configured to receive a subject′ limb) to prevent a substantive amount of water from leaking out. The seal 306 may comprise a sealing grommet or self-sealing grommet. Optionally, where the enclosure 302 may be configured with multiple openings (e.g., to enable a limb to be passed therethrough). Optionally, there are multiple seals on the entry opening.

For example, the enclosure opening may have two seals, one seal on the outer wall and one seal on the inner wall of the enclosure to inhibit water from flowing out when the person's limb is inserted into the opening through the outer grommet. The enclosure may further comprise an inlet port 308, configured to receive fluid or gel to fill the enclosure 302, and an outlet port 310, configured to drain the fluid or gel once the data gathering procedure is complete. One or both ports may comprise valves or seals to prevent fluid or gel from inadvertently draining, but which may be opened to permit fluid or gel to drain. The fluid or gel may be added via the inlet port 308 prior to subject's limb being inserted thereto, or after the subject's limb has been inserted into the enclosure 302.

Where a self-sealing grommet is used as a seal 306, the self-sealing grommet may comprise a flexible material, such as rubber or silicone, that is designed to compress and conform around the limb as it is passed through the opening. The self-sealing grommet may have a slit, a cross-slit, or opening in the center, which allows the limb to be easily inserted. As the limb is pushed through the grommet, the material of the grommet stretches and compresses around the limb, creating a tight seal.

The self-sealing grommet works by using the pressure of the limb to activate the seal. When the limb is removed, the grommet will return to its original shape and size, so that it may be used again.

The enclosure 302 may have one or multiple emitters and/or receiver 102, 104, 106 (respectively comprising an emitter or portion thereof 102A, 104A, 106A, and/or a receiver 102B, 104B, 106B) positioned on an internal wall of the enclosure and/or on an external wall of the enclosure. There may be one or multiple types of emitters or portions thereof, such as WiFi emitters 102A, sound emitters 104A, and/or light emitters 106A. There may be one or multiple emitters for a given emitter type, and there may be one or multiple receivers of a given receiver type. The emitters may be positioned to direct signal emissions at the limb.

For example, the WiFi emitters 102A may comprise an antenna that emits radio frequency (RF) signals in the 900 Mhz-5 Ghz range, although other frequencies may be used. Thus, the RF frequencies which may be used are much lower than the range 30 petahertz to 30 exahertz (3×10¹⁶ Hz to 3×10¹⁹ Hz) for x-rays, and have little or no known adverse impact to humans. Optionally, only the antenna is mounted on the enclosure 302, and the active security is positioned off of the enclosure 302.

With respect to sound frequencies, although ultrasound frequencies may be utilized, lower frequencies may be utilized as well. For example, sound signals emitted by the acoustic emitters 104A may be in the range of 20 Hz to 1 kHz, or 1 khz-20 khz, or 20 khz-100 khz may be used.

With respect to light emitted by light emitters 106A, different frequencies (such as thus discussed below) may be utilized depending on the depth of the subject structure. Light penetrates the skin to different depths depending on its wavelength or frequency. The amount of penetration is also affected by the type and thickness of the skin, as well as the absorption and scattering properties of the tissue.

Generally, light with shorter wavelengths (i.e., higher frequencies) has less penetration depth than light with longer wavelengths (i.e., lower frequencies). For example:
Ultraviolet (UV) light with wavelengths of less than 400 nanometers (nm) is absorbed by the top layer of skin (the epidermis) and may not penetrate deeper into the skin.
Visible light with wavelengths between 400 and 700 nm can penetrate the epidermis and reach the upper layer of the dermis (the papillary dermis).
Near-infrared (NIR) light with wavelengths between 700 and 1100 nm can penetrate deeper into the dermis and reach the lower layer of the dermis (the reticular dermis) and subcutaneous tissue.
Mid-infrared (MIR) light with wavelengths between 1100 and 3000 nm can penetrate even deeper into the skin and reach the fatty tissue beneath the skin.

Optionally, rather than or in addition to using light to penetrate the skin and to receive reflected light from internal structures, light reflected from the skin may be used by a machine learning engine to identify a body part (or other object), and the boundaries thereof.

Optionally, rather than using a purpose-built enclosure, an existing structure, commonly present in a house, may be utilized. For example, a bathtub, hot tub, or the like, filled with water (or optionally other fluid or gel) may be utilized in which a user may immerse themselves, where the water provides a good interface to project sensor signals into the user's body (e.g., substantially all of the body) or a portion thereof. The sensors (e.g., 102, 104, 106) may be affixed to one or more bathtub walls, such as the interior sidewalls or bottom interior wall (e.g., using hooks suction cups, magnets, and/or adhesives). Optionally, the sensors may be affixed to a rail system which may be placed in the bathtub. The sensors may be at a fixed location or may be transported (e.g., via an electric motor) up and down the rail. The rail may be free floating, may rest on the bathtub floor, or may be affixed to a bathtub wall (e.g., via suction cups and/or adhesive). The signal reflections from the user may be used to generate an inferred image as similarly described elsewhere herein.

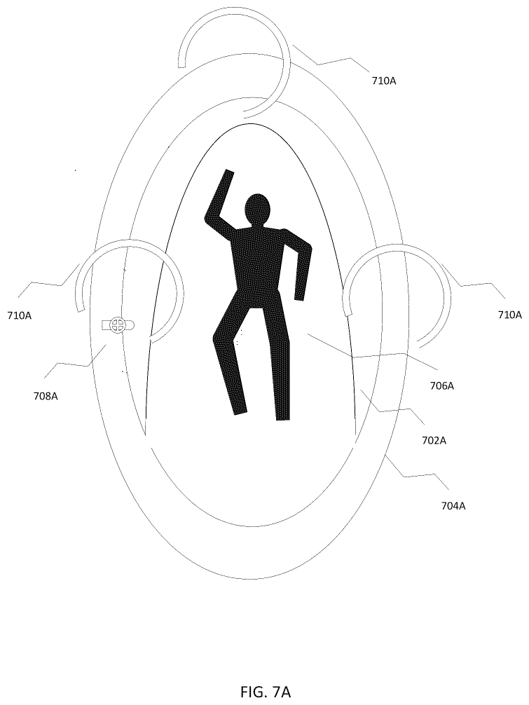

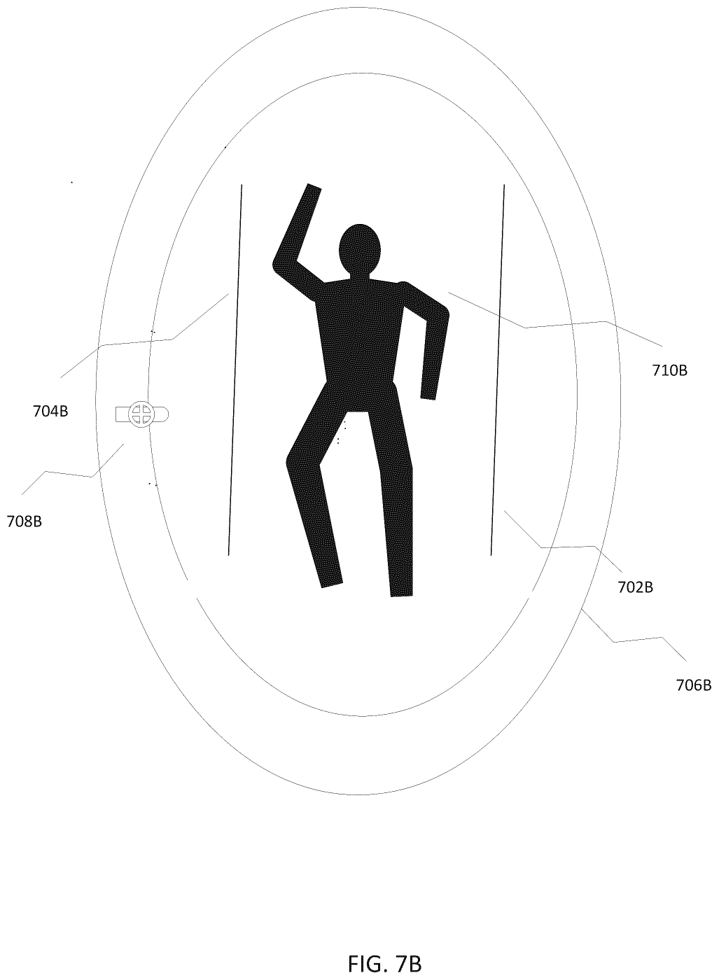

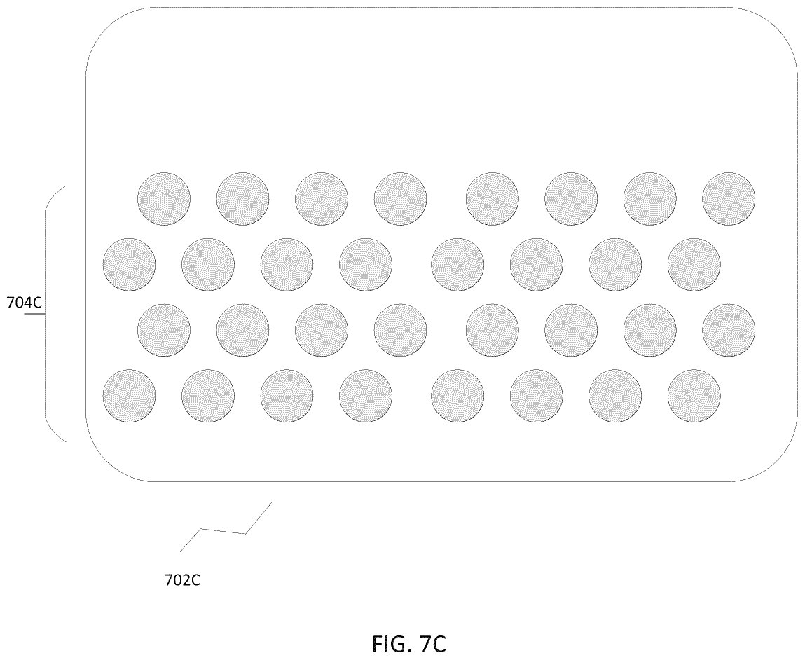

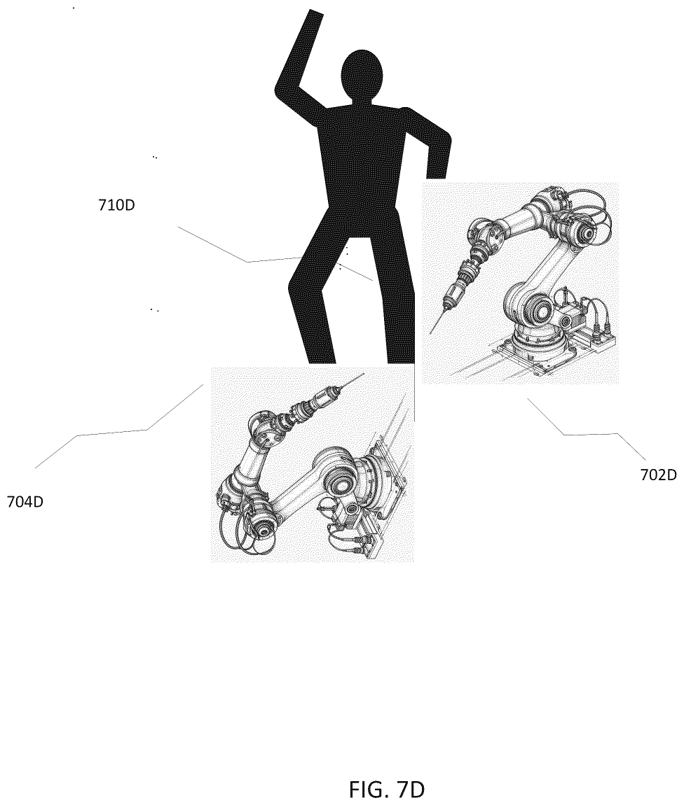

Referring now to FIG. 7A, an example implementation using a bathtub 704A (which may be a hot tub) is illustrated. A structure 702A (e.g., a u-shaped structure, such as an elliptically-shaped structure) may be placed in the bathtub 704A. The structure 702A may comprise a rigid structure and/or a flexible, spring-like structure. The structure 702A may comprise metal, plastic, and/or other material. The sensors (e.g., 102, 104, 106) may be disposed along the structure 702A, directing signals through the water towards the subject 706A. There may be one or more transmitters and one or more receivers for a given sensor type. The number of transmitters may be different than the number of receivers for a given sensor type. The bathtub may be filled with a fluid, such as water from spigot 708A, and/or a gel. The structure 702A may be removably affixed (e.g., using hooks, suction cups, magnets, and/or adhesives) to one or more bathtub sidewalls and/or the bathtub bottom wall. For example, hooks 710A may be mounted to each arm of the structure 702A and/or the u-shaped end of the structure 702A. A given hook may then be placed to hang on the nearest bathtub sidewall.

Referring now to FIG. 7B, another example implementation using a bathtub 706B (which may be a hot tub) is illustrated. Two structures 702B, 704B may be placed in the bathtub 704A, positioned to be on either side of a subject 710B. The structures 702B, 704B may comprise rigid structures and/or a flexible structures. The structures 702B, 704B may comprise metal, plastic, and/or other material. The sensors (e.g., 102, 104, 106) may be disposed along structures 702B, 704B, directing signals through the water towards the subject 710B. There may be one or more transmitters and one or more receivers for a given sensor type. The number of transmitters may be different than the number of receivers for a given sensor type. Optionally, one of the structures 702B, 704B may comprise all the transmitters, and the other one of the structures 702B, 704B may comprise all the receivers. Optionally, each of the structures 702B, 704B may include transmitters and receivers. The bathtub may be filled with a fluid, such as water from spigot 708B, and/or a gel. The structures 702B, 704B may be removably affixed (e.g., using hooks as illustrated in FIG. 7A, suction cups, magnets, and/or adhesives) to opposing bathtub sidewalls and/or the bathtub bottom wall.

Optionally, instead of in addition to having the removable structures discussed above with respect to FIG. 7A, 7B, the bathtub may be special-built or modified to include one or more of the sensors (e.g., 102, 104, 106) discussed herein. For example, with reference to FIG. 7C, the sensors 704C may be fixedly glued on, molded on, or otherwise affixed to one or more bathtub walls 702C (e.g., one or more bathtub sidewalls and/or the bottom wall).

Referring now to FIG. 7D, another example implementation using robot arms 702D, 704D is illustrated. The robot arms 702D, 704D may be positioned to be on either side of a subject 710D (e.g., a person's leg). The robot arms 702D, 704D may comprise sensor mounts at respective distal ends. The robot arms 702D, 704D may be controlled by a robot controller. There may be one or more transmitters and one or more receivers for a given sensor type (e.g., ultrasound, optical, RF, sound sensors and/or other sensor types disclosed herein) mounted to a given robot arm. The number of transmitters may be different than the number of receivers for a given sensor type. Optionally, one of the robot arms 702D, 704D may have mounted thereto all the transmitters, and the robot arm 704D may comprise all the receivers. Optionally, each of the robot arms 702D, 704D may have transmitters and receivers mounted thereto. Optionally, as described elsewhere herein, only one robot arm is used to position one or more sensors (e.g., emitters) and the other sensors (e.g., corresponding receivers) are fixed.

A robot arm may comprise a series of rigid links connected by joints, forming a kinematic chain. The joints may comprise revolute (rotational) joints and/or prismatic (linear) joints. At the end of the robot arm is the or end-effector (e.g., one or more sensor emitters and/or receivers). The robot arm may comprise one or more actuators (e.g., electric motors, hydraulic cylinders, and/or pneumatic actuators) that provide the power to drive the motion of the robot arm. A given joint of the robot arm may be actuated to enable controlled movement in multiple degrees of freedom.

The robot arm controller (not shown) may comprise a computer system and may be configured to manage the movement, position, and interactions of one or more robotic arms. The controller may comprise hardware interfaces that communicate with the robot arm's hardware components, such as motors, actuators, sensors, and/or other peripherals. The hardware interface may translate commands from the controller software into signals that control the physical motion of the arm. The controller may execute one or more motion control algorithms that determine how the robot arm moves in response to input commands. For example, the motion control algorithms may calculate the motor commands used to achieve desired positions, velocities, and accelerations while considering factors such as kinematics, dynamics, and constraints of the robot arm.

The controller may comprise kinematic models of the robotic arm, describing the relationships between joint angles or positions and the end-effector's position and orientation. The models are optionally used to calculate the inverse kinematics, enabling the controller to translate desired end-effector positions into joint motions. The controller may comprise and execute trajectory planning algorithms configured to generate smooth and efficient paths for the robot arm to follow while avoiding obstacles and respecting physical constraints. The trajectory planning algorithms may consider factors such as joint limits, velocity and acceleration limits, and collision avoidance to plan safe and optimal trajectories.

The controller may comprise and execute feedback control loops continuously monitor the robot arm's state and adjust its motion in real-time to maintain the desired positioning. The feedback control loops may use sensor feedback, such as position encoders and/or vision systems, to correct errors and deviations from the desired trajectory.

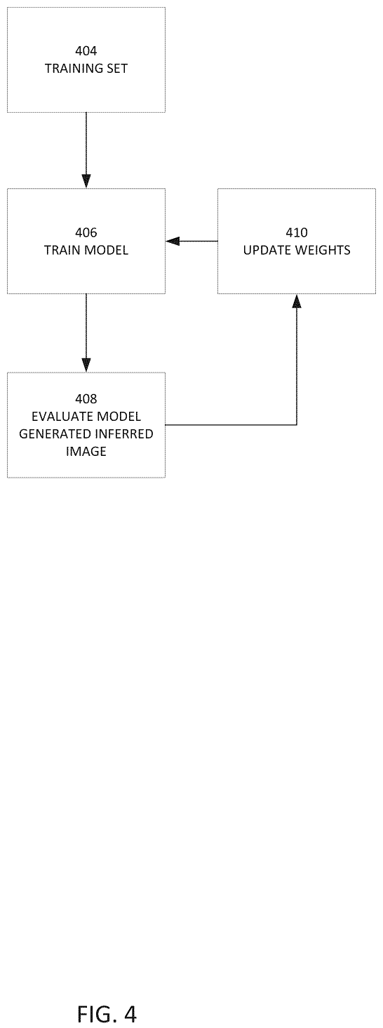

Referring now to FIG. 4, an example training process is illustrated. The process may optionally be executed using systems and devices disclosed herein (e.g., as illustrated in FIGS. 1-3). Not all states of the process need to be performed.

At block 404, a large set of training data is accessed (e.g., of previously generated images of an object).

Learning engines, such as neural networks, can synthesize an image from signals (e.g., RF, light, and/or sound signals) using a technique called signal-to-image synthesis.

For example, the training may comprise supervised learning. During training, the network is trained on a dataset of signals (e.g., reflected paired RF, sound, and/or light signals) and image examples corresponding to what the signals represent. At block 406, the network learns to associate the signals with the corresponding images by minimizing a loss function that measures the difference between the generated image and the real image.

In particular, the learning engine uses these pairs to learn the relationship between the reflected signals and the corresponding images. It does this by iteratively adjusting its internal parameters to minimize the difference between its predicted, synthesized images and the true images in the training set.

As discussed above, the network may comprise multiple layers of interconnected nodes, where a given layer may apply a transformation to its inputs. During training, the network receives a set of reflected signals as input and generates an image as output. At block 408, the generated inferred/predicted image is evaluated. The difference between this generated image and the true image is used to calculate a loss function, which measures how well the network is performing. At block 410, the network uses backpropagation to adjust its internal parameters to minimize this loss function.

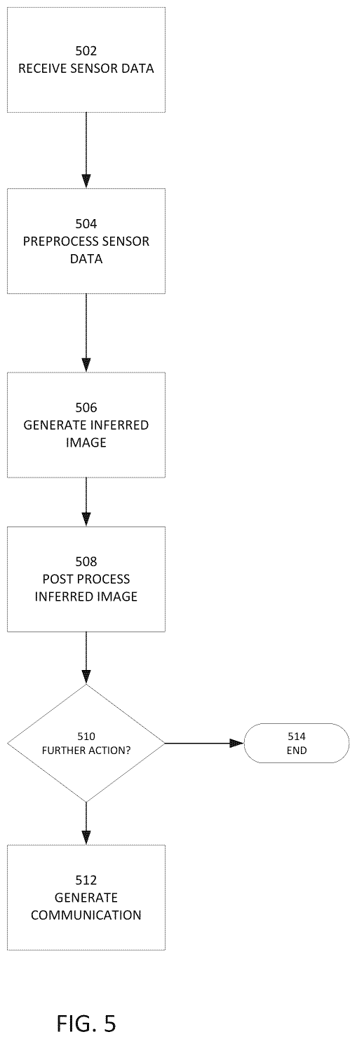

The foregoing process may be repeated multiple times, with the network gradually improving its performance as it learns to better approximate the relationship between the reflected signals and the corresponding images. Once training is complete, the network can be used to synthesize/predict images from reflected signals that it has not seen before, by applying the learned transformation to new inputs (e.g., via the example process illustrated in FIG. 5).

Referring now to FIG. 5, an example process is illustrated of generating an inferred image. At block 502, sensor data is received. For example, the sensor data may correspond to digitized sensor receiver data, which may comprise data corresponding to detected signals reflected by a subject. The sensor data may correspond to reflected RF (e.g., WiFi) signals, reflected acoustic signals, and/or reflected light signals as similarly discussed elsewhere herein.

At block 504, the received digitized sensor data may be preprocessed to enhance its suitability for being used by a learning engine. As similarly discussed elsewhere herein, the preprocessing may comprise performing denoising, interpolation, registration, windowing, and/or normalization. At block 506, an inferred image is predicted by a trained learning engine. Optionally, the learning engine may be instructed as to what the object is (e.g., an arm or leg) for which the image is to be generated. Optionally, the learning image may determine (e.g., from one or more of the reflected signal types, such as from the reflected light) what the object is.

At block 508, the inferred image may be post-processed. For example, the postprocessing may comprise contrast enhancement and/or image histogram adjustment to improve the visibility of significant of the image features. Optionally, image fusion may be used to combine images generated from different signal types into a composite image, which can provide a more complete and accurate picture of the subject. In addition, in order to align images from different sensors or different time periods, image registration techniques may optionally be used. Such image registration techniques may comprise adjusting the position, rotation, and scaling of images to ensure that they line up correctly. Optionally, feature extraction techniques may be utilized to isolate certain features and enhance their visibility, making it easier to identify and analyze them.

At block 510, based at least in part on an automated analysis of the generated inferred image, a determination may be made to whether an action should be taken. The automated analysis may identify a body part type, abnormal masses, fractures, blood clots, and/or the like. Optionally, the analysis may compare the newly generated inferred image with an historical inferred image of the same body part to determine if there are significant differences.

One or more techniques may be used to detect changes in inferred images over time. For example, image differencing may be utilized. Image differencing involves subtracting two images to obtain a third image that highlights the differences between the images.

By way of further example, an optical flow process may be utilized to detect changes over time in images. Optical flow is a technique that tracks the motion of pixels between two images of the same subject. By analyzing the motion of the pixels, regions that have changed over time may be detected.

By way of yet further example, a background subtraction process may be utilized, wherein a static background image is subtracted from a sequence of images to obtain a foreground mask. The foreground mask highlights the regions of the image that have changed over time, enabling detection of moving objects or changes in the foreground subject.

By way of still further example, deep learning techniques, such as convolutional neural networks (CNNs), may be trained and utilized to detect changes in images over time. The CNN may learn to identify features and patterns in the images that indicate changes, providing more accurate and robust change detection.

A combination of techniques may be used to improve the accuracy and reliability of the results.

For example, if some type of non-typical irregularity in a body part is detected (and/or if a difference relative to an older inferred image was detected), at block 512 a communication comprising a textual description of the non-typical irregularity in the body part finding and/or the image differences, and/or a link to, or the actual inferred image. The communication may be provided to medical personnel who may then schedule the subject for further examination and/or enhanced imaging. The communication may also be provided to the user so that the user can contact their medical provider for further investigation. If no unusual and/or unexpected features were identified, the process may proceed to block 514, and the process may end.

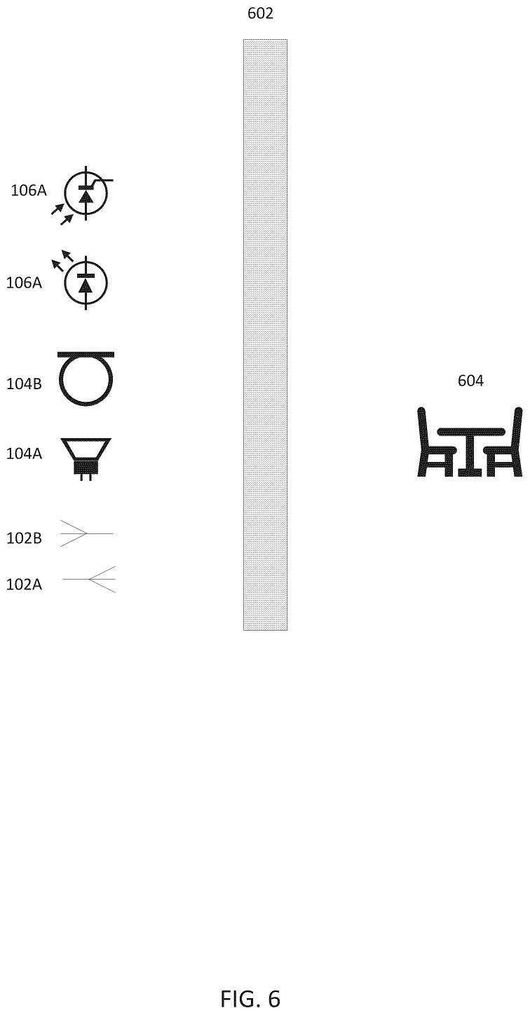

Although several examples have been described that relate to the human body, as also described herein the disclosed systems and processes may be utilized with respect to objects in general, including inanimate objects. For example, as illustrated in FIG. 6, one or more of the sensors 102A, 102B, 104A, 104B, 106A, 106B may be used to detect (e.g., using reflected RF and/or sound signals) objects, such as a table and/or chairs 604, behind an optically opaque wall 602. As similarly described above, the reflected signals may be used by a trained learning engine (e.g., inferred image synthesis engine) to generate an inferred image. Optionally, a trained learning engine (e.g., a CNN) may be utilized to identify one or more objects in the inferred image and to generate a message identifying the one or more objects.

Thus, methods and systems are described that utilize signals to identify an object, and/or to identify features of an object, using very low impact, low risk signals.

An aspect of the present disclosure relates to methods and systems configured to access a learning engine trained to generate an inferred image from a combination of reflections of different signal types, the different signal types comprising RF (e.g., WiFi), light, and/or sound signals. RF (e.g., WiFi), light, and/or sound signals are caused to be directed at a subject from a RF (e.g., WiFi) transmitter; a light emitter, and a sound generator, respectively. Respective receivers are used to receive reflections of the RF (e.g., WiFi), light, and/or sound signals from an object. The trained learning engine to generate an inferred image of an internal structure of the object from the received reflections of the RF (e.g., WiFi), light, and/or sound signals.

An aspect of the present disclosure relates to a system, comprising: at least one processing device operable to: access a learning engine trained to generate an inferred image from a combination of reflections of different signal types, the different signal types comprising WiFi, light, and sound signals; cause WiFi, light, and sound signals to be directed at a subject from a WiFi transmitter; a light emitter, and a sound generator, respectively; receive, via respective receivers, reflections of the WiFi, light, and/or sound signals from an object; use the trained learning engine to generate an inferred image of an internal structure of the object from the received reflections of the WiFi, light, and/or sound signals; analyze the generated inferred image; and based at least in part on the analysis of the generated inferred image, take a first action.

Optionally, the learning engine comprises an input layer, an output layer, and one or more hidden layers. Optionally, the learning engine comprises a generator and a discriminator. Optionally, the system further comprises an enclosure having at least a portion of the WiFi transmitter, the light emitter, and/or the sound generator and positioned thereon, the enclosure having an opening configured to receive the object, a grommet positioned at the opening, and a fluid inlet configured to receive fluid. Optionally, the system is configured to compare a plurality of inferred images and to identify differences between the plurality of inferred images. Optionally, the object comprises an inanimate object. Optionally, the object comprises an animate object. Optionally, the system is configured to detect signals reflected from the object, wherein an optically opaque wall separates the object from the WiFi transmitter; the light emitter, and the sound generator.

An aspect of the present disclosure relates to a system, comprising: at least one processing device operable to: access a learning engine trained to generate an inferred image from a combination of reflections of different signal types, the different signal types comprising RF (e.g., WiFi), light, and/or sound signals; cause RF, light, and/or sound signals to be directed at a subject from a RF transmitter; a light emitter, and a sound generator, respectively; receive, via respective receivers, reflections of the RF, light, and/or sound signals from an object; use the trained learning engine to generate an inferred image of an internal structure of the object from the received reflections of the RF, light, and/or sound signals.

Optionally, the learning engine comprises an input layer, an output layer, and one or more hidden layers. Optionally, the learning engine comprises a generator and a discriminator. Optionally, the system further comprises an enclosure having at least a portion of the WiFi transmitter, the light emitter, and/or the sound generator and positioned thereon, the enclosure having an opening configured to receive the object, a grommet positioned at the opening, and a fluid inlet configured to receive fluid. Optionally, the system is configured to compare a plurality of inferred images and to identify differences between the plurality of inferred images. Optionally, the object comprises an inanimate object. Optionally, the object comprises an animate object. Optionally, the system is configured to detect signals reflected from the object, wherein an optically opaque wall separates the object from the WiFi transmitter; the light emitter, and the sound generator.

An aspect of the present disclosure relates to a computer-implemented method, the method comprising: accessing a learning engine trained to generate an inferred image from a combination of reflections of different signal types, the different signal types comprising RF (e.g., WiFi), light, and/or sound signals; causing RF, light, and/or sound signals to be directed at a subject from a RF transmitter; a light emitter, and a sound generator, respectively; receiving, via respective receivers, reflections of the RF, light, and/or sound signals from an object; using the trained learning engine to generate an inferred image of an internal structure of the object from the received reflections of the RF, light, and/or sound signals.

Optionally, the learning engine comprises an input layer, an output layer, and one or more hidden layers. Optionally, the learning engine comprises a generator and a discriminator. Optionally, at least a portion of the RF transmitter, the light emitter, and/or the sound generator are positioned on an enclosure, the enclosure having an opening configured to receive the object, a grommet positioned at the opening, and a fluid inlet configured to receive fluid. Optionally, the method further comprises comparing a plurality of inferred images and to identify differences between the plurality of inferred images. Optionally, the object comprises an inanimate object. Optionally, the object comprises an animate object. Optionally, an optically opaque wall separates the object from the RF transmitter; the light emitter, and/or the sound generator.

The methods and processes described herein may have fewer or additional steps or states and the steps or states may be performed in a different order. Not all steps or states need to be reached. The methods and processes described herein may be embodied in, and fully or partially automated via, software code modules executed by one or more general purpose computers. The code modules may be stored in any type of computer-readable medium or other computer storage device. Some or all of the methods may alternatively be embodied in whole or in part in specialized computer hardware. The systems described herein may optionally include displays, user input devices (e.g., touchscreen, keyboard, mouse, voice recognition, etc.), network interfaces, etc.

The results of the disclosed methods may be stored in any type of computer data repository, such as relational databases and flat file systems that use volatile and/or non-volatile memory (e.g., magnetic disk storage, optical storage, EEPROM and/or solid state RAM).

The various illustrative logical blocks, modules, routines, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “may,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

While the phrase “click” may be used with respect to a user selecting a control, menu selection, or the like, other user inputs may be used, such as voice commands, text entry, gestures, etc. User inputs may, by way of example, be provided via an interface, such as via text fields, wherein a user enters text, and/or via a menu selection (e.g., a drop down menu, a list or other arrangement via which the user can check via a check box or otherwise make a selection or selections, a group of individually selectable icons, etc.). When the user provides an input or activates a control, a corresponding computing system may perform the corresponding operation. Some or all of the data, inputs and instructions provided by a user may optionally be stored in a system data store (e.g., a database), from which the system may access and retrieve such data, inputs, and instructions. The notifications/alerts and user interfaces described herein may be provided via a Web page, a dedicated or non-dedicated phone application, computer application, a short messaging service message (e.g., SMS, MMS, etc.), instant messaging, email, push notification, audibly, a pop-up interface, and/or otherwise.

The user terminals described herein may be in the form of a mobile communication device (e.g., a cell phone), laptop, tablet computer, interactive television, game console, media streaming device, head-wearable display, networked watch, etc. The user terminals may optionally include displays, user input devices (e.g., touchscreen, keyboard, mouse, voice recognition, etc.), network interfaces, etc.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A system, comprising:
at least one processing device operable to:
access a learning engine trained to generate an inferred image from a combination of reflections of different signal types, the different signal types comprising WiFi, light, and sound signals;
cause WiFi, light, and sound signals to be directed at a subject from a WiFi transmitter; a light emitter, and a sound generator, respectively;
receive, via respective receivers, reflections of the WiFi, light, and/or sound signals from an object;
use the trained learning engine to generate an inferred image of an internal structure of the object from the received reflections of the WiFi, light, and/or sound signals;
analyze the generated inferred image; and
based at least in part on the analysis of the generated inferred image, take a first action.

2. The system as defined in claim 1:
wherein the learning engine comprises an input layer, an output layer, and one or more hidden layers.

3. The system as defined in claim 1:
wherein the learning engine comprises a generator and a discriminator.

4. The system as defined in claim 1, the system further comprising an enclosure having at least a portion of the WiFi transmitter, the light emitter, and/or the sound generator and positioned thereon, the enclosure having an opening configured to receive the object, a grommet positioned at the opening, and a fluid inlet configured to receive fluid.

5. The system as defined in claim 1, wherein the system is configured to compare a plurality of inferred images and to identify differences between the plurality of inferred images.

6. The system as defined in claim 1, wherein the object comprises an inanimate object.

7. The system as defined in claim 1, wherein the object comprises an animate object.

8. The system as defined in claim 1, wherein the system is configured to detect signals reflected from the object, wherein an optically opaque wall separates the object from the WiFi transmitter; the light emitter, and the sound generator.

9. The system as defined in claim 1, wherein at least one receiver and/or at least one emitter is mounted to and positionable by a robotic arm.

10. A system, comprising:
at least one processing device operable to:
access a learning engine trained to generate an inferred image from a combination of reflections of different signal types, the different signal types comprising RF, light, and/or sound signals;
cause RF, light, and/or sound signals to be directed at a subject from a RF transmitter; a light emitter, and a sound generator, respectively;
receive, via respective receivers, reflections of the RF, light, and/or sound signals from an object;
use the trained learning engine to generate an inferred image of an internal structure of the object from the received reflections of the RF, light, and/or sound signals.

11. The system as defined in claim 10:
wherein the learning engine comprises an input layer, an output layer, and one or more hidden layers.

12. The system as defined in claim 10:
wherein the learning engine comprises a generator and a discriminator.

13. The system as defined in claim 10, the system further comprising an enclosure having at least a portion of the RF transmitter, the light emitter, and/or the sound generator and positioned thereon, the enclosure having an opening configured to receive the object, a grommet positioned at the opening, and a fluid inlet configured to receive fluid.

14. The system as defined in claim 10, wherein the system is configured to compare a plurality of inferred images and to identify differences between the plurality of inferred images.

15. The system as defined in claim 10, wherein the object comprises an inanimate object.

16. The system as defined in claim 10, wherein the object comprises an animate object.

17. The system as defined in claim 10, wherein the system is configured to detect signals reflected from the object, wherein an optically opaque wall separates the object from the RF transmitter; the light emitter, and the sound generator.

18. The system as defined in claim 9, wherein at least one receiver and/or at least one emitter is mounted to and positionable by a robotic arm.

19. A computer-implemented method, the method comprising:
accessing a learning engine trained to generate an inferred image from a combination of reflections of different signal types, the different signal types comprising RF, light, and/or sound signals;
causing RF, light, and/or sound signals to be directed at a subject from a RF transmitter; a light emitter, and a sound generator, respectively;
receiving, via respective receivers, reflections of the RF, light, and/or sound signals from an object;
using the trained learning engine to generate an inferred image of an internal structure of the object from the received reflections of the RF, light, and/or sound signals.

20. The computer-implemented as defined in claim 19:
wherein the learning engine comprises an input layer, an output layer, and one or more hidden layers.

21. The computer-implemented as defined in claim 19:
wherein the learning engine comprises a generator and a discriminator.

22. The computer-implemented as defined in claim 19, wherein at least a portion of the RF transmitter, the light emitter, and/or the sound generator are positioned on an enclosure, the enclosure having an opening configured to receive the object, a grommet positioned at the opening, and a fluid inlet configured to receive fluid.

23. The computer-implemented as defined in claim 19, the method further comprising comparing a plurality of inferred images and to identify differences between the plurality of inferred images.

24. The computer-implemented as defined in claim 19, wherein the object comprises an inanimate object.

25. The computer-implemented as defined in claim 19, wherein the object comprises an animate object.

26. The computer-implemented as defined in claim 19, wherein an optically opaque wall separates the object from the RF transmitter; the light emitter, and/or the sound generator.

27. The computer-implemented as defined in claim 19, the method further comprising positioning the RF transmitter; the light emitter, and/or the sound generator/or one or more RF, light, and/or sound receivers using a robotic arm.
